(19) United States
(12) Patent Application Publication
(10) Pub. No.: US 2024/0081724 A1
(43) Pub. Date: Mar. 14, 2024

(54) WEARABLE POINT-OF-CARE DEVICES FOR ASSESSING IMMUNE ACTIVITY FROM INTERSTITIAL FLUID AND METHODS OF USE THEREOF

(71) Applicants: Jason Michael STROHMAIER, Philadelphia, PA (US); Ryan Casey BOUTWELL, Henrico, VA (US); Michael A. DANIELE, Cary, NC (US)

(72) Inventors: Jason Michael STROHMAIER, Philadelphia, PA (US); Ryan Casey BOUTWELL, Henrico, VA (US); Michael A. DANIELE, Cary, NC (US)

(21) Appl. No.: 17/769,505

(22) PCT Filed: Oct. 16, 2020
(86) PCT No.: PCT/US2020/056042

§ 371 Date: Apr. 15, 2022

Related U.S. Application Data
(60) Provisional application No. 62/916,183, filed on Oct. 16, 2019.

(51) Int. Cl. A61B 5/00; A61B 5/145

(52) U.S. Cl. CPC A61B 5/412; A61B 5/14514; A61B 5/14546; A61B 5/685; A61B 2503/40

(57) ABSTRACT

This disclosure provides a wearable point-of-care (POC) device for assessing immune activity in a subject in need thereof, the device comprising: a microneedle patch capable of making conformal contact with a skin of the subject, the microneedle patch comprising a plurality of microneedles: a lateral flow test strip comprising a plurality of channels, each of the channels in the plurality of channels comprising control and capture antibodies for an analyte in a plurality of analytes, wherein the control and capture antibodies are deposited along a length of the channel such that upon exposure to the analyte a number of visible spots in the channel is correlated with a concentration of the analyte; and a means for pumping an interstitial fluid from the plurality of microneedles and through each of the channels in the plurality of channels when the device is on the skin of the subject.

### CROSS-REFERENCE TO RELATED APPLICATIONS

This application claims priority to and the benefit of co-pending U.S. provisional application entitled “WEARABLE POINT-OF-CARE DEVICES FOR ASSESSING IMMUNE ACTIVITY FROM INTERSTITIAL FLUID AND METHODS OF USE THEREOF” having Ser. No. 62/916,183 filed Oct. 16, 2019.

### TECHNICAL FIELD

The present disclosure generally relates to point-of-care medical devices and systems.

### BACKGROUND

The future battlespace presents unique challenges to military medicine. Multi-domain operations (MDO) require sophisticated medical systems to operate with extended time-on-station without replenishment or support. Complex wounds and polytrauma encountered in the operational environment expose warfighters to opportunistic pathogens and present unique challenges in military medicine. Of great concern is the anticipated loss of the “golden hour” for medical evacuation, increasing the need to care for the casualties at or near the point of injury. This new paradigm increases the risks of wound infections, physiological decompensation, and sepsis. Military medicine requires fast-acting diagnosis of infections to best assess the operational environment, maintain the most ready and healthy force, and care for and rehabilitate injured warfighters.

Battlefield casualties are difficult to clean and maintain, and wounds are frequently complicated by infection. The Infectious Disease Clinical Research Program (IDCRP) at the Uniformed Services University reported in case studies IDCRP-044 and IDCDP-077 that bone infections from open fractures and invasive fungal infections are common in complex dismounted blast trauma.² Other researchers are investigating biofilm inhibition techniques against opportunistic bacteria such as the antibiotic-resistant Pseudomonas aeruginosa, which is a common cause of infection in burn patients.³ Skin and soft tissue infections (SSTI) such as those caused by community-acquired methicillin-resistant Staphylococcus aureus (CA-MRSA) are a growing threat to US military superiority. Better characterization of their progression is required for improved management of combat trauma-related infections in future wars. The DOD's Trauma Infectious Disease Outcomes Study identified that from 2009-2012 over a third of patients with blast trauma developed at least one trauma-related infection, with extremity wounds being the most frequent.⁴⁻⁶ Infections, which lead to sepsis, threaten both physiological and psychological components of military performance as well as mission success.

Each hour of delay in sepsis treatment leads to an increased odds of mortality.⁷ Sepsis is the result of a systemic inflammatory response to an infectious burden. However, infection can be difficult to detect in certain settings, leading to treatment delays. Specifically, sepsis recognition is challenging in low resource settings and in patients with wound infections. Areas with low-resources, or austere settings, have low availability of healthcare personnel, basic clinical laboratory infrastructure, and treatments. Further delays can occur from difficulty obtaining intravenous access for blood sampling. This clinical picture is more opaque with skin wounds. The healing process of wounds and burns lead to increased levels of inflammation that can be difficult to differentiate from an acute life-threatening infection. These components lead to great challenges for warfighters that may be susceptible to sepsis due to skin infections or other causes.

There remains a need for improved systems and methods for rapid point-of-care diagnosis of infections that overcome the aforementioned deficiencies.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 is (right) a view of an exemplary wearable point-of-care device according to a first aspect of the disclosure and (left) a close-up image of the microneedle array capable of conformal skin contact and sampling.

FIGS. 2A-2B include images of an exemplary nanocomposite microneedles that swell with fluid (FIG. 2A) and showing that fluid can be extracted by integrated paper microfluidics (FIG. 2B) according to a second aspects of the disclosure.

FIGS. 3A-3B depict an example design of LFT for analyzing ISF (FIG. 3A) and LFT layout for quantitative “binning” of analyte concentrations (FIG. 3B). The inset of FIG. 3B depicts example photograph of LFT array. As increasing concentrations are present in the LFT, more test spots increase in color intensity.

FIG. 4 includes data of quantified analyte concentration in simulated and real human dermal ISF, including lactate and glucose. This figure also shows a comparison of controlled vs in vitro measured glucose with good correlation, as well as human skin during ISF extraction.

FIG. 5 shows a microneedle applicator and microneedle patch and human skin stained with Gentian Violet to image microneedle perforation after use. Needle tip diameter is approximately 10 um and application area is 1 sq. cm.

FIG. 6 shows a bandage embodiment of the DermiSense Microneedle patch, allowing placement on the skin, insertion of the microneedles, and straightforward removal for storage and shipping, if required.

### DETAILED DESCRIPTION

In various aspects, point-of-care devices and methods of use are provided for directly monitoring immune function. The devices are inexpensive and do not require a power source, thereby making them particularly useful in the battlefield and in other difficult and/or remote terrains. Directly monitoring immune function and response can provide an early indicator of infection. At the earliest stage of infection, cytokines act to trigger increased production of white blood cells. During advanced infection, cytokines control innate and adaptive immunity and regulate both lymphocyte proliferation and apoptosis. Mapping the concentration dynamics of circulating cytokines to identify early indicators of decompensation and infection would better inform military medicine in austere environments.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, techniques of nanotechnology, organic chemistry, material science and engineering and the like, which are within the skill of the art. Such techniques are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

Better surveillance of humoral immunity by mapping circulating cytokines in the prodromal and active phases of infection could enable the detection of such biofilm producing opportunistic invaders before consequences become dire. Complex extremity injury where polymicrobial infection is likely requires new tools for military health decision makers to optimize warfighter performance and recovery.

Despite vigorous efforts in the research community, commercially available sepsis tests are all based on the culture of blood samples, and they require between 6 hours and 5 days for confirmation of diagnosis. Although some care protocols recommend immediate sepsis treatment with antimicrobials following symptom presentation (i.e. before blood culture confirmation), a reduction in patient survival in some studies has questioned the wisdom of acting in advance of test result confirmation.

Infectious disease detection relies too frequently on self-reported symptoms of the patient. In fast paced and demanding environments symptom report can be delayed, increasing infection consequence for individual warfighters and their teams; reducing readiness and increasing risk to the mission. Once symptoms present, diagnosis is often determined (or confirmed) with invasive blood draw and time-intensive blood culturing. Not only does this delay the determination of appropriate care, it requires special training, is painful, cannot be easily self-applied, and opens the body for additional foreign assault to the patient. Easier, earlier monitoring of the immune response of exposed individuals will reduce the spread of infection and improve outcomes for those who are infected. The ability to detect changes in immune activity would save thousands of infections annually, millions of dollars and lost hours of productivity, and many lives.

A zero-power, shelf-stable, painless, simple-to-use, and easily interpreted way to monitor immune response would add significant value in the fight to limit the consequences of infection. DermiSense is developing this solution. This disclosure provides a skin patch with tiny needles that painlessly collect ISF for testing. ISF lacks cells and clotting agents, which can complicate blood analysis, thereby making it an attractive target for POC testing. With continuous testing, a personalized approach to sepsis recognition can be possible through detecting changes from a patient's baseline.

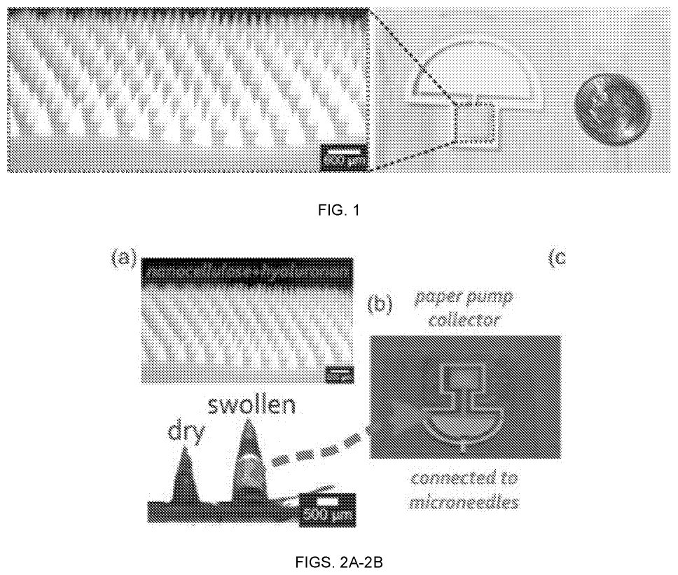

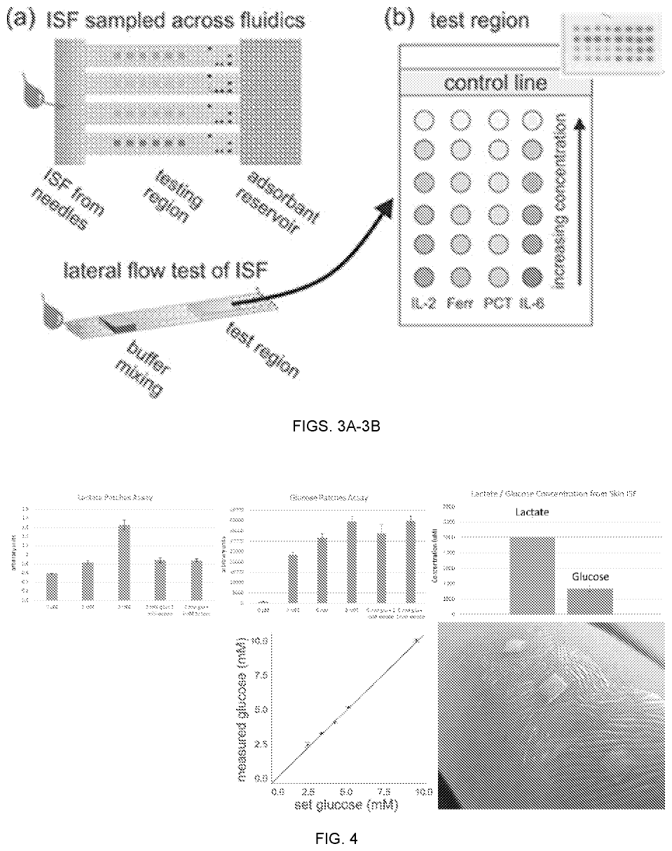

This disclosure provides nanocomposite microneedle platform, as shown in FIG. 1. Unlike most microneedle technologies, the provided microneedle test strip is flexible, porous, and allows the passage of moisture and oxygen to the skin. These key attributes allow for better conformity on the user's skin and accelerated ISF extraction rates of the same sample volume compared to other non-porous, non-hydrophilic microneedle platforms. The quality of microneedle product is directly dependent upon the mixture and quality of the extraction, compounding, cleaning, drying, processing, and forming of the microneedles themselves. Optimizing the materials processing enables fine control over the nanocomposite, modifying flexibility/stiffness, porosity/wick-ability, wettability, and fluid flow. Optimizing the materials and processing ensures the transport properties of key analytes to ensure are consistent across devices and absorbed and flow at a well-characterized rate for analyses.

This disclosure provides a wearable point-of-care (POC) device for assessing immune activity in a subject in need thereof, the device comprising: a microneedle patch capable of making conformal contact with a skin of the subject, the microneedle patch comprising a plurality of microneedles configured to extend below a stratum corneum of the skin and into an epidermis or dermis of the skin when placed in conformal contact with the skin; a lateral flow test strip comprising a plurality of channels, each of the channels in the plurality of channels comprising control and capture antibodies for an analyte in a plurality of analytes, wherein the control and capture antibodies are deposited along a length of the channel such that upon exposure to the analyte a number of visible spots in the channel is correlated with a concentration of the analyte; and a means for pumping an interstitial fluid from the plurality of microneedles and through each of the channels in the plurality of channels when the device is on the skin of the subject. The means for pumping can be one that requires no power, e.g. an evaporative pad or absorptive reservoir that drives the ISF transport, e.g. through wicking.

The microneedle patch can be made from a variety of biocompatible materials. In some aspects, applicants have found that microneedle patches made from microbial nanocellulose and hyaluronan allow for superior mechanical and chemical properties. The microneedles can be made to have an average length of about 40 μm to about 1000 μm or about 40 μm to about 600 μm. The microneedle patch can be made to have appropriate mechanical properties such as a failure stress greater than about 1 MPa, e.g. about 1 MPa to about 19 MPa or about 19 MPa for making strong conformal contact. The materials can also be made to be porous to allow for air to reach the skin through the patch, improving the ability to maintain conformal contact.

The devices can be used to analyze a large variety of analytes. The analytes can include one or more of the analytes listed in Table 1. In some aspects, the analytes include all 28 candidate biomarkers in Table 1 and the plurality of channels in the lateral flow test strip comprise a separate channel for each of the 28 analytes. In some aspects, the analytes include IL-1a, IL-1β, and IL-6, MIP-1α, IFNγ, AST, ALT, G-CSF, GM-CSF, ora combination thereof.

### PROPHETIC EXAMPLES

Now having described the aspects of the present disclosure, in general, the following prophetic Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of aspects of the present disclosure.

DermiSense has demonstrated the ability to synthesize and control the yield of microbial nanocellulose. Using both wood-based and microbial nanocellulose, bio-nanocomposite films can be fabricated for wearable biosensors and bioelectronics.⁸⁻¹⁰

Nanocomposite and Hydrogel Microneedles; Thin-Film Nanocellulose Microfluidics. Microneedle patches have potential to overcome the limitations of blood and sweat analyses by continuous ISF testing.¹¹ Following the work by Prausnitz et al.¹² and Donnelly et al.¹³, we construct hydrogel microneedles, but use nanocellulose as a composite support to ensure mechanical stability and improve absorption of fluids. We have integrated these nanocomposite microneedles with a paper pump for ISF collection¹⁴ and demonstrated the absorbance and pumping of fluid from model skin (seen in FIG. 2). A membrane was punctured by the microneedle to simulate skin penetration; when the microneedles reach the fluid reservoir, the dried microneedles absorbed nearly 600% of their starting volume, which is approximately 3× greater than crosslinked-hyaluronic acid needles. The fluid is wicked into the paper for further analysis.

### Prophetic Example 1. Optimization and Analyses of ISF Extraction with Microneedles

Based on nanocellulose-hyaluronan nanocomposites, the materials can be optimized and the microneedle-microfluidic patches can be fabricated that can penetrate skin, extract and transport ISF.

In Example 1, we develop and characterize nanocomposite microneedle patches that extract ISF, partition the ISF sample, and then analyze the constituency of the extracted ISF. The nanocomposite microneedle patches c (i) determine the desired formulation for long-term penetration of the skin and (ii) maximize the extracted volume of ISF. The integrated microfluidics may be characterized to optimize the ability to wick fluids from microneedle patches. The nanocomposite structure and processing parameters can be correlated to performance of the microneedle patch.

### Example 1.1: Optimizing Material Composition and Processing

Achieving ISF extraction and transport requires developing nanocomposites that exhibit (i) high mechanical durability to maintain skin insertion and (ii) high volumetric swelling. During this phase, one can synthesize nanocomposite microneedles incorporating (i) microbial nanocellulose and (ii) hyaluronan, ideal materials for developing microneedles. Both form high-water content hydrogels, are biocompatible, and possess sufficient chemical flexibility to provide chemical modification.¹⁵⁻¹⁷ Hyaluronan is often utilized for dissolvable microneedles. 18-2° Independent processing steps control the microneedle size (mold geometry) and density (material loading and drying process). The nanocomposite microneedles can be fabricated by casting aqueous suspensions of nanocellulose and hyaluronan into silicone molds. Formulations can range, for example, from 0-5% nanocellulose and 1-5 wt. % total solids. The microneedles can then be dried by either lyophilization, vacuum oven, or evaporation at elevated temperature. After drying, the microneedles can be crosslinked. Control microneedles, i.e. cross-linked hyaluronan, can also be fabricated for comparison.

The fundamental swelling mechanisms of various microneedle formulation recipes developed during this phase can be investigated by measuring the change in physical dimensions of the swelling microneedles, and by measuring the total amount of water imbibed by the microneedles over time. For swelling studies, microneedles can be exposed to water, phosphate buffered saline (PBS), or artificial ISF.²³ For compressive testing, failure stress of =19 MPa can be targeted to achieve penetration of the skin.³⁰⁻³²

This systematic investigation of the underlying microneedle processing and the resulting determination of optimal formulation recipe can enable a more comprehensive strategy for manufacturing scalability.

### Example 1.2 In Vivo Murine Models

A complex biological system is often required to study the myriad host-pathogen interactions associated with infectious diseases. One can use the microneedle patches on small animal models for extraction and assay of target immune system biomarkers in ISF Of particular interest in the collected ISF are signal molecules called lymphokines, pro-inflammatory lymphokines such as the interleukin 1 family as well as interleukin 2 (IL-2), which signals increased activity of T cell white blood cells (WBC). WBC are active participants in the adaptive immune system and important indicators of the host's immune system health. In addition to IL-2, the collected dermal ISF can be analyzed for all lymphokine concentrations, seeking a comprehensive review of immune system activity (see table below). Present literature has not yet reported an investigation looking specifically for IL-2 in healthy ISF, though other interleukins and higher molecular weight proteins have been detected. The clinical course of sepsis and other infection is rapid, consisting of rise in cytokine level, a hypodynamic cardiovascular state, exuberant rise in serum cytokine levels and progression toward death within 12 to 24 h.^{38, 39} Microneedle patches with optimum swelling can be placed on the dorsum for 30 minutes, and exchanged every other hour for 24 hours. Samples can be analyzed for the panel of cytokines described in Table 1.

Measuring 21 candidate biomarkers in ISF can provide additional impact (Table 1). For example, real time mapping of pro-inflammatory interleukins such as IL-1α, IL-1β, and IL-6 combined with other inflammatory cytokines like MIP-1α and IFNγ could provide early indication of state changes in host immune activity. Liver enzymes such as AST/ALT ratio and chemokines such as G-CSF and GM-CSF can also elucidate host immune response activity. Mapping cytokine activity during the course of immune response can identify relevant biomarkers for prodromal and acute infectious activity.

TABLE 1
Exemplary Analytes of interest

| Analyte | Physiologic context |
|---|---|
| ALT | Hepatic injury marker (standard blood panel) |
| AST | Hepatic injury marker (standard blood panel) |
| FGF | Acute dermal injury factor |
| G-CSF | Colony stimulating factor (granulocyte production |
| | and release) |
| GM-CSF | Colony stimulating factor (granulocyte/macrophage |
| | production and release) |
| IFN-gamma | Cytokine that is critical for innate and adaptive |
| | immunity |
| IGF | Acute dermal injury factor |
| IL-13 | Central mediator induced by allergic inflammation |
| IL-1a | Pro-inflammatory cytokine |
| IL-1b | Pro-inflammatory cytokine |
| IL-2 | Regulates leukocyte activity |
| IL-21 | Cytokine associate with mechanical skin injury |
| IL-6 | Pro-inflammatory cytokine |
| IL-8 | Neutrophil directing chemokine |
| TGF-beta | Multifunctional cytokine impacting immune response |
| TNF-alpha | Pro-inflammatory; acute phase reaction |
| Procalcitonin | Reflects systemic response to bacterial infection and |
| | severity⁵¹ |
| Human neutrophil | Present in bacterial infection to differentiate from viral |
| lipocalin (HNL) | infection⁵² |
| TNF-related | May be uniquely present during host response to viral |
| apoptosis- | infection⁵³ |
| inducing ligand | |
| (TRAIL) | |
| IP-10 | May be useful in the determination of viral infections |
| C-Reactive | Higher in bacterial infections than viral⁵⁴ |
| Protein | |

### Prophetic Example 2. In Vivo Evaluation and Interrogation of Human ISF

We can evaluate the performance of the proposed microneedle patch for extraction and assay of dermal ISF. Moreover, we can comprehensively characterize the composition of extracted ISF and compare to venous and capillary blood. This specific task guides the future development of all non-blood biomarker assays, while identifying a potential footprint of infection to engineer the necessary sensors.

### Example 2.1 In Vivo Evaluation and Interrogation of Human ISF

The results of the described human subject evaluation can be used as supporting evidence to proceed with a formal, interventional Clinical Trial.

Example 2.1 encompasses a benchmarking effort for the technology space. While ISF measurements of glucose or electrolytes concentration have been described, infectious biomarker concentrations in ISF have not been well defined.⁵⁵

The correlation between ISF biomarker levels, plasma biomarker levels, and physiological status is largely unknown. A comprehensive characterization of cytokine and chemokine profiles in ISF, in comparison to venous and capillary blood, is needed.

Sample Collection. After cleaning the skin site with an isopropyl alcohol pad, the microneedle patch can be initially placed at the time of enrollment into the parent protocols. The patch can be applied for 30 minutes. ISF samples can be collected in multiple (6 microneedle patches per time point). Microneedle patches with or without integrated assays, or fully-integrated microsystems can be applied to the patient to collect and assay ISF.

Determine Levels of Protein and Metabolite Profiles. Sample analysis begins with a preparation step that isolates the analytes from their collected environment. The process for removal of the analytes from the microneedle patch can involve a washing of the patch to extract the analytes without removal or leaching of components from the patch that could interfere with the analysis. Most importantly, we can measure and normalize by the total protein concentration of each ISF sample. We can use the Bicinchoninic Acid (BCA) method, as it is insensitive to components commonly found in antibody array buffers. Most samples cannot need to be concentrated. If concentration is required, we can use a spin-column concentrator with a chilled centrifuge. All samples collected can be stored under conditions in which analyte stability has been demonstrated. This ISF sample and blood samples can be frozen at −80° C., where they may be stored under the same conditions until removed for processing and analysis. Antibody array kits or magnetic bead-based immunoassays may be performed to measure analyte levels. Blood samples can use serum or plasma for analyses.

### Example 2.2. Additional Demonstrations of Capability

One example application of the zero power DermiSense microneedle patch for immune response monitoring is in detection of sepsis from infections in austere environments. By evaluating differences across patients and across time, we can identify relevant biomarkers to detect acute infection with zero-power tests to facilitate the prevention or early treatment of sepsis.

Example 2.2.1. We can determine the range of select biomarker levels in dermal ISF in septic and healthy subjects.

Example 2.2.2. We can determine the correlation and performance of ISF biomarker levels compared to serum/plasma levels of biomarkers.

Example 2.2.3: We can evaluate within subject changes in ISF levels during acute infection (between 0 h, 6 h, and 24 h) and the healthy phase (between 0 h and 12 months) for associations with 28-day mortality and sepsis severity.

Analyses for Example 2.2: We can determine the range and distribution of biomarker levels measured by optical density in dermal ISF at time 0 hours (during acute septic phase). Among participants who screen negative for ongoing infection, the range and distribution of biomarker levels in dermal ISF and in serum at 6 months and 12 months can be determined.

### Prophetic Example 3. Integration of Lateral Flow Assay (Proof-of-Principle)

For zero-power, wireless, wearable analyses of the ISF, we can integrate a quantitative lateral flow test, though many types of zero power simple visual tests could be used. This test can enable the real time detection of immune response and infection status with zero applied power. Results can be easily interpreted by non-medically trained personnel.

### Example 3: Integration of Lateral Flow Test

ISF that passes through our optimized microneedle collection platform can be detected using colorimetric sensing in a lateral flow immunoassay (similar to home pregnancy test kits). Colorimetric sensing is a well-established technique that requires zero-power. As the target analyte collects in the sensing location, increasing analyte concentration increases the color intensity of the sensor (e.g. for red-tagged molecules the sensor becomes deeper and deeper red with increasing analyte concentration).

### Example 3.1: Design and Control Testing of Lateral Flow Test (LFT)

In Example 3.1, we can design, fabricate, and test a cytokine detection assay suitable for detection of multiple biomarkers for improved diagnosis of infection and sepsis. While many LFT have been created for biomarker quantification,⁵⁸⁻⁶² we can base our LFT on a selection of biomarkers presented in Table 1. A schematic illustration of the quantitative LFT is presented in FIG. 3. As the ISF flows across the LFT, detection and capture antibodies are present to quantify select analytes. By controlling the deposition of the control and capture antibodies, we can be able to correlate the number of visible spots (developed after exposure to the analyte) to the concentration of the analyte. For example, 1-10 μM would result in 1 visible spot, 10-20 μM would results in 2 visible spots, etc. This design of LFT has been named “lateral flow microarray.” The panel of detection and capture agents provides for higher sensitivity compared to LFT employ mixed antibodies in a conventional test line. A very similar approach has been demonstrated for the diagnosis of immune response in a microarray for 15 targets.⁶³

As an example, we can fabricate an LFT for the following analytes: IL-2, IL-6, and Procalcitonin. IL-2 regulates leucocyte activity; Procalcitonin has been used to distinguish bacterial from viral infections. A sandwich immunoassay based on antigen-antibody reaction can be employed on the LFT with a label of dyed microparticles attached to the detection antibodies. We have previously demonstrated the regioselective functionalization of detection agents in LFTs,⁶⁴ which improved selectivity and sensitivity. Different color microparticles can be used for each analyte. Detection and capture antibodies are available for all targets, and the viability of LFT for these analytes have been demonstrated in some capacity (always operated in serum or blood).⁶⁵⁻⁶⁸ A series of reference standards can be tested at 0, 0.5, 2, 10, 20, and 40 ng/mL by diluting the analyte (100 ng/mL) with the dilution buffer. Three quality controls can be set at 0, 2, and 10 ng/mL by diluting the capture agents with analyte-free serum or simulated ISF.

The LFT can be carried out in vitro, across an array of analyte concentrations, detection and capture antibody conditions, and microfluidic papers.

Based on the above optimization experiments, we can prepare small batch products with the optimized parameters. All of the test strips can be dried, cut and placed in disposable cassettes. All of the products can be stored with high stability and performance. After initial storage period of 90 days, the LFT can be tested again for sensitivity and specificity.

### Example 3.2: Integration of LFT with Microneedle Patch

A custom lateral flow test strip holder can be designed to stabilize a microneedle array, allowing the sampled fluid to transported form the nanocomposite microneedles to the sample collection pad of the LFT.

It should be emphasized that the above-described embodiments of the present disclosure are merely possible examples of implementations, and are set forth only for a clear understanding of the principles of the disclosure. Many variations and modifications can be made to the above-described embodiments of the disclosure without departing substantially from the spirit and principles of the disclosure. All such modifications and variations are intended to be included herein within the scope of this disclosure.

### Example 3.3: Integration of Microneedle Patch with Smart Adhesive

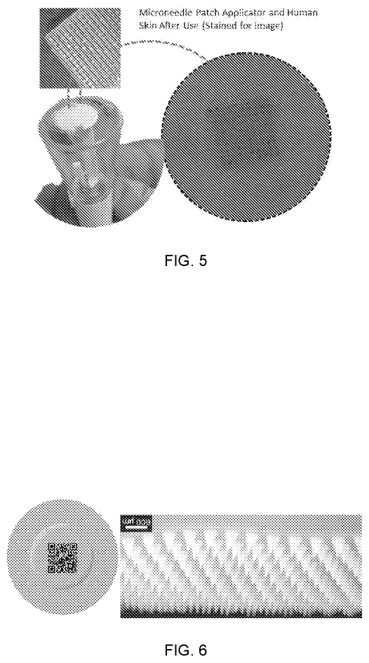

The microneedle patch may be integrated into an adhesive for insertion and extraction on skin, shown in FIG. 5 and FIG. 6. This adhesive may be colored, opaque, or transparent to allow for visualization or other transmission of LFT or other sensor results conveyed from underneath the adhesive surface. The patch may enable sensing by externally-applied power, such as near field communication (NFC), integrated into the adhesive. The patch may be marked with serialized information such as a Quick Response (QR) code to enable patch identification by a data aggregator such as a smartphone, and convey other relevant patch use data.

### REFERENCES

1. B. Q. Tran, P. R. Miller, R. M. Taylor, G. Boyd, P. M. Mach, C. N. Rosenzweig, J. T. Baca, R. Polsky and T. Glaros, J Proteome Res, 2018, 17, 479-485.
2. I. D. C. R. P. (IDCRP), Trauma Related Infections, https://www.idcrp.org/research-area/trauma-related-infections, (accessed Oct. 4, 2019).
3. T. Lenzmeier, N. Mudaliar, J. Stanbro, C. Watters, A. A. Ahmad, M. P. Simons, G. Ventolini, J. Zak, J. Colmer-Hamood and A. N. Hamood, Journal of Medical Microbiology, 2019, DOI: 10.1099/jmm.0.001066, 13.
4. K. Mende, L. Stewart, F. Shaikh, W. Bradley, D. Lu, M. R. Krauss, L. Greenberg, Q. L. Yu, D. M. Blyth, T. J. Whitman, J. L. Petfield and D. R. Tribble, Diagn Micr Infec Dis, 2019, 94, 173-179.
5. D. R. Tribble, N. G. Conger, S. Fraser, T. D. Gleeson, K. Wilkins, T. Antonille, A. Weintrob, A. Ganesan, L. J. Gaskins, P. Li, G. Grandits, M. L. Landrum, D. R. Hospenthal, E. V. Millar, L. H. Blackbourne, J. R. Dunne, D. Craft, K. Mende, G. W. Wortmann, R. Herlihy, J. McDonald and C. K. Murray, J Trauma, 2011, 71, S33-S42.
6. D. R. Tribble, M. R. Krauss, C. K. Murray, T. E. Warkentien, B. A. Lloyd, A. Ganesan, L. Greenberg, J. H. Xu, P. Li, M. L. Carson, W. Bradley, A. C. Weintrob, I. D. C. R. Progra and T. I. D. O. Stu, Surg Infect, 2018, 19, 494-503.
7. D. C. Angus and T. van der Poll, New Engl J Med, 2013, 369, 840-851.
8. J. D. Yuen, S. A. Walper, B. J. Melde, M. A. Daniele and D. A. Stenger, Sci Rep, 2017, 7, 40867.
9. M. A. Daniele, A. J. Knight, S. A. Roberts, K. Radom and J. S. Erickson, Adv Mater, 2015, 27, 1600-1606.
10. S. Jandhyala, S. A. Walper, A. A. Cargill, A. Ozual and M. A. Daniele, Smart Biomedical and Physiological Sensor Technology Xiii, 2016, 9863.
11. P. P. Samant and M. R. Prausnitz, P Natl Acad Sci USA, 2018, 115, 4583-4588.
12. A. V. Romanyuk, V. N. Zvezdin, P. Samant, M. I. Grenader, M. Zemlyanova and M. R. Prausnitz, Anal Chem, 2014, 86, 10520-10523.
13. E. Caffarel-Salvador, A. J. Brady, E. Eltayib, T. Meng, A. Alonso-Vicente, P. Gonzalez-Vazquez, B. M. Torrisi, E. M. Vicente-Perez, K. Mooney, D. S. Jones, S. E. Bell, C. P. McCoy, H. McCarthy, J. C. McElnay and R. F. Donnelly, Plos One, 2015, 10, e0145644.
14. B. M. Cummins, R. Chinthapatla, B. Lenin, F. S. Ligler and G. M. Walker, Technology, 2017, 5, 21-30.
15. K. J. De France, T. Hoare and E. D. Cranston, Chem. Mat., 2017, 29, 4609-4631.
16. C. B. Highley, G. D. Prestwich and J. A. Burdick, Curr Opin Biotech, 2016, 40, 35-40.
17. J. A. Burdick and G. D. Prestwich, Adv Mater, 2011, 23, H41-H56.
18. J. Arya, S. Henry, H. Kalluri, D. V. McAllister, W. P. Pewin and M. R. Prausnitz, Biomaterials, 2017, 128, 1-7.
19. Y. C. Kim, J. H. Park and M. R. Prausnitz, Adv Drug Deliver Rev, 2012, 64, 1547-1568.
20. J. C. Yu, Y. Q. Zhang, Y. Q. Ye, R. DiSanto, W. J. Sun, D. Ranson, F. S. Ligler, J. B. Buse and Z. Gu, P Natl Acad Sci USA, 2015, 112, 8260-8265.
21. A. Sannino, S. Pappada, M. Madaghiele, A. Maffezzoli, L. Ambrosio and L. Nicolais, Polymer, 2005, 46, 11206-11212.
22. K. Tomihata and Y. Ikada, J Biomed Mater Res, 1997, 37, 243-251.
23. A. H. Bretag, Life Sci Pt 1 Physi, 1969, 8, 319-&.
24. O. Okay, Springer Ser Chem Se, 2009, 6, 1-14.
25. F. J. Holly, J Biomed Mater Res, 1975, 9, 315-326.
26. F. J. Holly and M. F. Refojo, Abstr Pap Am Chem S, 1975, 170, 102-102.
27. A. Al-Abboodi, J. Fu, P. M. Doran and P. P. Y. Chan, Biotechnol Bioeng, 2013, 110, 318-326.
28. J. D. Yuen, S. A. Walper, B. J. Melde, M. A. Daniele and D. A. Stenger, Sci Rep-Uk, 2017, 7.
29. S. Brunauer, P. H. Emmett and E. Teller, J Am Chem Soc, 1938, 60, 309-319.
30. P. Aggarwal and C. R. Johnston, Sensor Actuat B-Chem, 2004, 102, 226-234.
31. J. H. Park and M. R. Prausnitz, J Korean Phys Soc, 2010, 56, 1223-1227.
32. K. S. Anseth, C. N. Bowman and L. BrannonPeppas, Biomaterials, 1996, 17, 1647-1657.
33. S. A. Ranamukhaarachchi, S. Lehnert, S. L. Ranamukhaarachchi, L. Sprenger, T. Schneider, I. Mansoor, K. Rai, U. O. Hafeli and B. Stoeber, Sci Rep-Uk, 2016, 6.
34. M. J. Garland, K. Migalska, T. M. Tuan-Mahmood, T. R. R. Singh, R. Majithija, E. Caffarel-Salvador, C. M. McCrudden, H. O. McCarthy, D. Woolfson and R. F. Donnelly, Int J Pharmaceut, 2012, 434, 80-89.
35. K. Moronkeji, S. Todd, I. Dawidowska, S. D. Barrett and R. Akhtar, J Control Release, 2017, 265, 102-112.
36. E. Larraneta, S. Stewart, S. J. Fallows, L. L. Birkhauer, M. T. C. McCrudden, A. D. Woolfson and R. F. Donnelly, Int J Pharmaceut, 2016, 497, 62-69.
37. G. E. P. Box and K. B. Wilson, J Roy Stat Soc B, 1951, 13, 1-45.
38. J. A. Buras, B. Holzmann and M. Sitkovsky, Nat Rev Drug Discov, 2005, 4, 854-865.
39. E. A. Deitch, Shock, 1998, 9, 1-11.
40. K. M. Williams, D. Pallapies, B. A. Peskar, K. Brune and G. Geisslinger, Clin Pharmacol Ther, 1995, 57, 159-159.
41. K. T. Chou, S. C. Cheng, S. F. Huang, D. W. Perng, S. C. Chang, Y. M. Chen, H. S. Hsu and S. C. Hung, Sci Rep-Uk, 2019, 9.
42. J. A. Stortz, M. K. Hollen, R. B. Hawkins, M. C. Cox, D. C. Nacionales, R. Ungaro, M. L. Dirain, J. Rincon, Z. K. Wang, A. Bihorac, M. C. Lopez, H. V. Baker, S. C. Brakenridge, C. Leeuwenburgh, F. A. Moore, A. M. Mohr, L. L. Moldawer and P. A. Efron, Shock, 2019, 51, 131-132.
43. A. J. Lewis and M. R. Rosengart, Surg Infect, 2018, 19, 137-141.
44. A. J. Lewis, C. W. Seymour and M. R. Rosengart, Surg Infect, 2016, 17, 385-393.
45. A. J. Lewis, C. W. Seymour, D. Yuan and M. R. Rosengart, J Am Coll Surgeons, 2015, 221, S41-S41.
46. A. J. Lewis, D. Yuan, X. H. Zhang, D. C. Angus, M. R. Rosengart and C. W. Seymour, Crit Care Med, 2016, 44, E420-E431.
47. M. H. Ling and M. C. Chen, Acta Biomater, 2013, 9, 8952-8961.
48. S. Indermun, Y. E. Choonara, P. Kumar, L. C. du Toit, G. Modi, R. Luttge, M. Govender and V. Pillay, J Pharm Sci, 2017, 106, 1111-1116.
49. A. C. Lauer, J. T. Elder and N. D. Weiner, J Pharm Sci, 1997, 86, 13-18.
50. H. Kalluri, C. S. Kolli and A. K. Banga, Aaps J, 2011, 13, 473-481.
51. N. Brechot, G. Hekimian, J. Chastre and C. E. Luyt, Int J Antimicrob Ag, 2015, 46, S19-S24.
52. P. Venge, Upsala J Med Sci, 2018, 123, 1-8.
53. N. Cummins and A. Badley, Curr Mol Med, 2009, 9, 495-505.
54. K. Sasaki, I. Fujita, Y. Hamasaki and S. Miyazaki, J Infect Chemother, 2002, 8, 76-80.
55. P. P. Samant and M. R. Prausnitz, Proceedings of the National Academy of Sciences, 2018, 115, 4583-4588.
56. K. L. Schully, C. C. Young, M. Mayo, A. L. Connolly, V. Rigas, A. Spall, A. A. Chan, M. G. Salvador, J. V. Lawler, J. A. Opdyke, D. V. Clark and B. J. Currie, Clin Infect Dis, 2019, 69, 421-427.
57. V. Suttisunhaku, P. Hip, P. Ouch, P. Ly, C. Supaprom, A. Rachmat, M. Prouty, A. Vaughn, A. Eltayeb, S. Kheng, D. V. Clark, J. V. Lawler, N. Chantratita, M. N. Burtnick, P. J. Brett and K. L. Schully, Am J Trop Med Hyg, 2018, 98, 1039-1045.
58. J. D. Bishop, H. V. Hsieh, D. J. Gasperino and B. H. Weigl, Lab on a Chip, 2019, 19, 2486-2499.
59. B. Ngom, Y. C. Guo, X. L. Wang and D. R. Bi, Anal Bioanal Chem, 2010, 397, 1113-1135.
60. C. Carrell, A. Kava, M. Nguyen, R. Menger, Z. Munshi, Z. Call, M. Nussbaum and C. Henry, Microelectron Eng, 2019, 206, 45-54.
61. O. Miocevic, C. R. Cole, M. J. Laughlin, R. L. Buck, P. D. Slowey and E. A. Shirtcliff, Front Public Health, 2017, 5.
62. M. Sajid, A. N. Kawde and M. Daud, J Saudi Chem Soc, 2015, 19, 689-705.
63. T. Chinnasamy, L. I. Segerink, M. Nystrand, J. Gantelius and H. A. Svahn, Analyst, 2014, 139, 2348-2354.
64. M. D. Wilkins, B. L. Turner, K. R. Rivera, S. Menegatti and M. Daniele, Sensing and bio-sensing research, 2018, 21, 46-53.
65. X. Y. Shao, C. R. Wang, C. M. Xie, X. G. Wang, R. L. Liang and W. W. Xu, Sensors, 2017, 17.
66. M. D. Wimmer, M. M. Ploeger, M. J. Friedrich, R. Bornemann, P. P. Roessler, S. Gravius and T. M. Randau, Technol Health Care, 2016, 24, 927-932.
67. Y. Wang, J. Y. Sun, Y. J. Hou, C. Zhang, D. W. Li, H. X. Li, M. F. Yang, C. D. Fan and B. L. Sun, Biosens Bioelectron, 2019, 141.
68. Z. D. Lu, D. O'Dell, B. Srinivasan, E. Rey, R. S. Wang, S. Vemulapati, S. Mehta and D. Erickson, P Natl Acad Sci USA, 2017, 114, 13513-13518.

1. A wearable point-of-care (POC) device for assessing immune activity in a subject in need thereof, the device comprising:
a microneedle patch capable of making conformal contact with a skin of the subject, the microneedle patch comprising a plurality of microneedles configured to extend below a stratum corneum of the skin and into an epidermis or dermis of the skin when placed in conformal contact with the skin;
a test strip comprising a plurality of channels, each of the channels in the plurality of channels comprising a sensor for an analyte in a plurality of analytes; and
a means for moving an interstitial fluid from the plurality of microneedles and through each of the channels in the plurality of channels when the device is on the skin of the subject.

2. The device according to claim 1, wherein at least one of the channels comprises control and capture chemistry for an analyte in a plurality of analytes, wherein the control and capture chemistries are deposited along a length of the channel such that upon exposure to the analyte a number of visible locations along the channel are correlated to a concentration or a type of the analyte.

3. The device according to claim 2, wherein the control and capture chemistry comprises antibodies, pH indicators, temperature indicators, electrochemical sensors, particle size filters, chemical binding sites, or combinations thereof.

4. The device according to claim 1, wherein the means for moving comprises microfluidic transport by wicking to an evaporative pad, an absorptive pad, or a combination thereof.

5. The device according to claim 1, wherein the plurality of analytes comprises one or more of the analytes listed in Table 1.

6. The device according to claim 1, wherein the plurality of analytes comprises all candidate biomarkers in Table 1 and the plurality of channels in the test strip comprise a separate channel for each of the analytes.

7. The device according to claim 1, wherein the plurality of analytes comprises IL-1α, IL-1β, and IL-6, MIP-1α, IFNγ, AST, ALT, G-CSF, GM-CSF, or a combination thereof.

8. The device according to claim 1, wherein the microneedles comprise microbial nanocellulose and hyaluronan.

9. The device according to claim 8, wherein pendant functional groups on the hyaluronan and/or nanocellulose are crosslinked.

10. The device according to claim 9, wherein the pendant functional groups are carboxyl groups on the hyaluronan that are crosslinked by diimide-coupling.

11. The device according to claim 1, wherein the microneedle patch has a failure stress of about 19 MPa.

12. The device according to claim 1, wherein the device comprises one or more electronic sensors that provide sensing capabilities based on the electrical signal.

13. The device according to any one of claims 1-12claim 1, wherein the device comprises one or more sensors that communicate a type or a concentration of the analyte through haptic feedback, visual display, sound, smell, or taste.

14. The device according claim 1, further comprising a communication means to communicate data on the types and concentrations of analytes.

15. The device according to claim 14, wherein the communication means is wired or wireless.

16. The device according to claim 14 or claim 15, wherein the device communicates with a smart phone, tablet, or other connected device that can generate additional data.

17. The device according to claim 1, wherein the channels are visible when the device is in use, and
wherein a color change can be analyzed via an external application on a smartphone or other connected device to analyze the results.

18. A method of assaying the immune activity of a subject, the method comprising
applying a device according to claim 1 to a skin of the subject, wherein the microneedle patch forms a conformal contact with the skin of the subject and the microneedles in the plurality of microneedles extend below a stratum corneum of the skin and into an epidermis or dermis of the skin;
allowing an interstitial fluid of the subject to be collected through the microneedles and to be moved across the test surface of the device;
analyzing the results from the test surface of the device to determine the immune activity of the subject.

19. The method according to claim 18, wherein the analyzing comprises observing one or more visible indicators in a channel of the test strip, wherein the one or more visible indicators correlate to an identity or a concentration of an analyte indicative of the immune activity of the subject.

20. The method according to claim 18 or claim 19, wherein the analyte comprises one or more analytes listed in Table 1.

21. The method according to claim 18, wherein the microneedle device includes unique identifying information to allow for serialization or unique identifying information detectable through handheld device or smartphone imaging; and
wherein the method comprises identifying the identifying information and at least one type or concentration of an analyte.

22. The method according to claim 18, wherein the analyzing comprises assessing non-visible markers or communicating electronically off of the patch to a connected device using a communication means.

23. The method according to claim 18, further comprising communicating a sensor status off-device to a data aggregator.

24. The method according to claim 18, further comprising communication through a handheld electronic device or smartphone system with standardized/quantified color measurement and collecting metadata to send population health information back to a central data repository.

25. The method according to claim 18, wherein the subject comprises a human subject or a nonhuman subject.

26. The method according to claim 18, further comprising evaluating a historical record of analyte concentration by collecting and analyzing the evaporative pad after removing the device.

27. The method according to claim 18, comprising integrating the output with other common wearable sensors selected from the group consisting of temperature, movement, electrochemical sensing, photoplethysmography, near infrared spectroscopy, and a combination thereof.

28. The method according to claim 18, comprising integrating the outputs of one microneedle device with the outputs of others to form a central database of immune activity or infection activity across a population of interest.

29. The device according to claim 1, wherein at least one of the channels comprises control and capture chemistry for an analyte in a plurality of analytes, wherein the control and capture chemistries are deposited in the channel such that upon exposure to the analyte a brightness or color change is correlated with a concentration or a type of the analyte;
a means for moving an interstitial fluid from the plurality of microneedles and through each of the channels in the plurality of channels when the device is on the skin of the subject.

30. The device according to claim 1, wherein one or more of the sensors is operated by externally applied power.

31. The device according to claim 1, wherein the microneedles in the plurality of microneedles comprise an average length of about 40 μm to about 1000 μm.
